(19) United States
(12) Patent Application Publication
(10) Pub. No.: US 2019/0040395 A1
(43) Pub. Date: Feb. 7, 2019

(54) MODULATORS OF KLK3 ERNA

(71) Applicant: Ionis Pharmaceuticals, Inc., Carlsbad, CA (US)

(72) Inventor: Susan M. Freier, San Diego, CA (US)

(73) Assignee: Ionis Pharmaceuticals, Inc., Carlsbad, CA (US)

(21) Appl. No.: 16/074,744

(22) PCT Filed: Feb. 16, 2017
(86) PCT No.: PCT/US17/18074

§ 371 Date: Aug. 1, 2018

Related U.S. Application Data
(60) Provisional application No. 62/297,063, filed on Feb. 18, 2016.

(51) Int. Cl. C12N 15/113; A61P 35/00

(52) U.S. Cl. CPC C12N 15/1137; A61P 35/00; C12N 2310/315; C12N 2310/341; C12N 2310/11; C12N 2310/3341

(57) ABSTRACT

The present embodiments provide methods, compounds, and compositions useful for inhibiting KLK3 eRNA expression, which may be useful for treating, preventing, or ameliorating cancer, such as prostate cancer.

### SEQUENCE LISTING

The present application is being filed along with a Sequence Listing in electronic format. The Sequence Listing is provided as a file entitled BIOL0290WOSEQ_ST25.txt, created on Feb. 13, 2017 which is 20 KB in size. The information in the electronic format of the sequence listing is incorporated herein by reference in its entirety.

### FIELD

The present embodiments provide methods, compounds, and compositions useful for inhibiting KLK3eRNA expression, which can be useful for treating, preventing, or ameliorating cancer, such as prostate cancer.

### BACKGROUND

Androgen receptor (AR) is a transcription factor implicated as a driver of prostate cancer. AR is activated by binding to its hormone ligands: androgen, testosterone, and/or DHT, which regulates the expression of several genes. Androgen deprivation therapy, also known as “chemical castration,” is a first-line treatment strategy against hormone-sensitive, androgen-dependent prostate cancer that reduces circulating androgen levels and thereby inhibits AR activity. However, androgen deprivation therapy frequently leads to the emergence and growth of “castration-resistant” advanced prostate cancer, in which AR signaling is reactivated independent of ligand binding.

Recent high-throughput transcriptomic analyses have revealed that eukaryotic genomes transcribe up to 90% of the genomic DNA. (The ENCODE Project Consortium. The ENCODE (ENCyclopedia of DNA Elements) Project. Science 2004; 306:636-640). Only 1-2% of these transcripts encode for proteins, whereas the vast majority are transcribed as non-coding RNAs (ncRNAs).

The majority of the non-protein-coding transcripts belong to the group of lncRNAs, which are considered as >200 nucleotides in length. Most lncRNAs are characterized by nuclear localization, low expression, low level of sequence conservation and are composed of both poly A+ and poly A− transcripts. (Kapranov P, et al., “RNA maps reveal new RNA classes and a possible function for pervasive transcription.” Science 2007; 316:1484-1488) (Wu Q, et al., “Poly A− transcripts expressed in HeLa cells.” PLoS One 2008; 3:e2803).

A subgroup of lncRNAs, termed enhancer RNAs (eRNAs), was recently reported to be transcribed from genomic enhancer regions. (Kim TK et al., “Widespread transcription at neuronal activity-regulated enhancers.” Nature 2010; 465:182-187) (De Santa F et al., “A large fraction of extragenic RNA pol II transcription sites overlap enhancers.” PLoS Biol 2010; 8:e1000384).

Kallikrein-related peptidase 3 (KLK3) encodes for prostate-specific antigen (PSA) and is a known AR-regulated gene. Transcription of the KLK3 upstream enhancer generates KLK3 enhancer RNA (KLK3 eRNA). Hsieh et al. Proc. Natl. Acad. Sci. USA 2014 May 20; 111(20):7319-24.

### SUMMARY

Embodiments provided herein are directed to compounds and compositions useful for inhibiting KLK3 eRNA expression, which can be useful for treating, preventing, ameliorating, or slowing progression of cancer, such as prostate cancer

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS number (ISIS #) indicate a combination of nucleobase sequence, chemical modification, and motif.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“5-methylcytosine” means a cytosine with a methyl group attached to the 5 position.

“About” means within ±10% of a value. For example, if it is stated, “the compounds affected about 70% inhibition of KLK3 eRNA”, it is implied that KLK3 eRNA levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Individual” means a human or non-human animal selected for treatment or therapy.

“Inhibiting the expression or activity” refers to a reduction or blockade of the expression or activity relative to the expression of activity in an untreated or control sample.and does not necessarily indicate a total elimination of expression or activity.

“internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“KLK3 eRNA” means the RNA transcript of the KLK3 enhancer. KLK3 eRNA is described, for example, in Hsieh et al. Proc. Natl. Acad. Sci. USA 2014 May 20; 111(20):7319-24, which is incorporated by reference herein in its entirety.

“KLK3 eRNA specific inhibitor” refers to any agent capable of specifically inhibiting KLK3 eRNA expression or activity at the molecular level. For example, KLK3 eRNA specific inhibitors include nucleic acids (including antisense compounds), peptides, antibodies, small molecules, and other agents capable of inhibiting the expression of KLK3 eRNA.

“Linked nucleosides” means adjacent nucleosides linked together by an internucleoside linkage.

“Modulating” refers to changing or adjusting a feature in a cell, tissue, organ or organism. For example, modulating KLK3 eRNA can mean to increase or decrease the level of KLK3 eRNA in a cell, tissue, organ or organism. A “modulator” effects the change in the cell, tissue, organ or organism. For example, a KLK3 eRNA compound can be a modulator that decreases the amount of KLK3 eRNA in a cell, tissue, organ or organism.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Prevent” refers to delaying or forestalling the onset, development or progression of a disease, disorder, or condition for a period of time from minutes to indefinitely.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Standard cell assay” means assay(s) described in the Examples and reasonable variations thereof.

“Targeting” means the specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Therapeutically effective amount” means an amount of a compound, pharmaceutical agent, or composition that provides a therapeutic benefit to an individual.

“Treat” refers to administering a compound or pharmaceutical composition to an animal in order to effect an alteration or improvement of a disease, disorder, or condition in the animal.

### Certain Embodiments

Certain embodiments provide methods, compounds and compositions for inhibiting KLK3 eRNA expression. Certain embodiments provide methods, compounds and compositions for inhibiting KLK3 eRNA expression in a cell.

Certain embodiments provide compounds targeted to a KLK3 eRNA. In certain embodiments, the KLK3 eRNA has the sequence set forth in GENBANK Accession No NT_011109.17_TRUNC_23609000_23621000 (incorporated by reference, disclosed herein as SEQ ID NO: 1). In certain embodiments, the KLK3 eRNA has the sequence set forth in SEQ ID NO: 2. In certain embodiments, KLK3 eRNA has the sequence described in in Hsieh et al. Proc. Natl. Acad. Sci. USA 2014 May 20; 111(20):7319-24, which is incorporated by reference herein in its entirety.

In certain embodiments, the compound is single-stranded. In certain embodiments, the compound is double-stranded.

In certain embodiments, a compound comprises or consists of a modified oligonucleotide 16 linked nucleobases in length having a nucleobase sequence comprising the sequence recited in SEQ ID NO: 3 (GAACCTTGGTTAGGCA), wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

In certain embodiments, a compound comprises or consists of a modified oligonucleotide 16 linked nucleobases in length having a nucleobase sequence comprising the sequence recited in SEQ ID NO: 4 (ATGGTGCTGGCCACAC), wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

In any of the foregoing embodiments, the compound or oligonucleotide can be at least 85%, at least 90%, at least 95%, at least 98%, at least 99%, or 100% complementary to KLK3 eRNA.

In any of the foregoing embodiments, the compound can be single-stranded. In certain embodiments, the compound comprises deoxyribonucleotides. In certain embodiments, the compound is double-stranded. In certain embodiments, the compound is double-stranded and comprises ribonucleotides.

### Certain Indications

Certain embodiments provided herein relate to methods of inhibiting KLK3 eRNA expression, which can be useful for treating, preventing, or ameliorating cancer, such as prostate cancer, in an individual, by administration of a compound that targets KLK3 eRNA. In certain embodiments, such a compound is a KLK3 eRNA specific inhibitor. In certain embodiments, the compound is an antisense compound, oligomeric compound, or oligonucleotide targeted to KLK3 eRNA.

In certain embodiments, a method of treating, preventing, or ameliorating cancer, such as prostate cancer, in an individual comprises administering to the individual a specific inhibitor of KLK3 eRNA, thereby treating, preventing, or ameliorating the disease. In certain embodiments, the KLK3 eRNA specific inhibitor is a compound targeted to KLK3 eRNA, such as an oligonucleotide targeted to KLK3 eRNA.

In certain embodiments, a method of inhibiting expression of KLK3 eRNA in an individual having, or at risk of having cancer, such as prostate cancer, comprises administering a KLK3 eRNA specific inhibitor to the individual, thereby inhibiting expression of KLK3 eRNA in the individual. In certain embodiments, administering the inhibitor inhibits expression of KLK3 eRNA in the prostate. In certain embodiments, the individual has, or is at risk of having cancer, such as prostate cancer. In certain embodiments, the KLK3 eRNA specific inhibitor is a compound targeted to KLK3 eRNA, such as an oligonucleotide targeted to KLK3 eRNA. In certain embodiments, a method of inhibiting expression of KLK3 eRNA in a cell comprises contacting the cell with a KLK3 eRNA specific inhibitor, thereby inhibiting expression of KLK3 eRNA in the cell. In certain embodiments, the cell is a cancer cell, such as a prostate cancer cell. In certain embodiments, the cell is in the prostate. In certain embodiments, the cell is in the prostate of an individual who has, or is at risk of having cancer, such as prostate cancer.

Certain embodiments are drawn to a KLK3 eRNA specific inhibitor for use in treating cancer, such as prostate cancer. In certain embodiments, the disease is cancer, such as prostate cancer.

Certain embodiments are drawn to use of a KLK3 eRNA specific inhibitor for the manufacture or preparation of a medicament for treating cancer, such as prostate cancer. Certain embodiments are drawn to use of a KLK3 eRNA specific inhibitor for the preparation of a medicament for treating cancer, such as prostate cancer.

In any of the foregoing methods or uses, the compound targeted to KLK3 eRNA or specific inhibitor of KLK3 eRNA comprises or consists of a modified oligonucleotide 16 linked nucleobases in length having a nucleobase sequence comprising the sequence recited in SEQ ID NO: 3 or SEQ ID NO: 4, wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

In any of the foregoing methods or uses, the compound can be administered parenterally. For example, in certain embodiments the compound can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Hybridization

In some embodiments, hybridization occurs between a compound disclosed herein and a KLK3 eRNA nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a KLK3 eRNA nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### A. Modified Nucleosides

Modified nucleosides comprise a modified sugar moiety or a modified nucleobase or both a modifed sugar moiety and a modified nucleobase.

1. Modified Sugar Moieties

In certain embodiments, modified oligonucleotides comprise 4′-CH(CH₃)—O-2′ (referred to as “constrained ethyl” or “cEt” when in the S configuration) sugar modifications. cEt modifications are described in U.S. Pat. No. 7,399,845; U.S. Pat. No. 7,741,457; U.S. Pat. No. 8,022,193; and U.S. Pat. No. 7,569,686, which are incorporated by reference herein in their entireties.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, compounds targeted to a KLK3 eRNA comprise one or more modified nucleobases. In certain embodiments, the modified nucleobase is 5-methylcytosine. In certain embodiments, each cytosine is a 5-methylcytosine.

3. Modified Internucleoside Linkages

The naturally occuring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

In certain embodiments, compounds targeted to a KLK3 eRNA comprise one or more modified internucleoside linkages. In certain embodiments, the modified internucleoside linkages are phosphorothioate linkages. In certain embodiments, each internucleoside linkage of a modified oligonucleotide is a phosphorothioate (“P=S”) internucleoside linkage.

4. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are 16 linked nucleosides in length comprising:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Human KLK3 eRNA in C4-2 Cells

Antisense oligonucleotides were designed targeting human KLK3 eRNA and were tested for their effects on KLK3 eRNA levels in vitro. The antisense oligonucleotides were tested in a series of experiments that had similar culture conditions. The results for each experiment are presented in separate tables shown below. Cultured C4-2 human prostate cancer cells at a density of 30,000 cells per well were transfected using electroporation with 10,000 nM antisense oligonucleotide. After a treatment period, RNA was isolated from the cells and KLK3 eRNA levels were measured by quantitative real-time PCR. Human primer probe set RTS4882 (forward sequence GGAGAATTGCCTCCCAACAC, designated herein as SEQ ID NO: 5; reverse sequence TTAATGGTGGAACGTTGAGACTGT, designated herein as SEQ ID NO: 6; probe sequence TTCAGCCAGAGCCTTCCACCCTTG, designated herein as SEQ ID NO: 7) was used to measure RNA levels. KLK3 eRNA levels were adjusted according to total RNA content, as measured by RIBOGREEN®. Results are presented as percent inhibition of KLK3 eRNA, relative to untreated control cells.

The newly designed chimeric antisense oligonucleotides were designed as 3-10-3 (S)-cET gapmers. The gapmers are 16 nucleosides in length, wherein the central gap segment consists of ten 2′-deoxynucleosides and is flanked by wing segments on both the 5′ direction and on the 3′ direction consisting of three nucleosides per wing. Each nucleoside in the 5′ wing segment and each nucleoside in the 3′ wing segment has an (S)-cEt modification. The internucleoside linkages throughout each gapmer are phosphorothioate linkages. All cytosine residues throughout each gapmer are 5-methylcytosines. “Start site” indicates the 5′-most nucleoside to which the gapmer is targeted in the human enhancer gene sequence. “Stop site” indicates the 3′-most nucleoside to which the gapmer is targeted in the human enhancer gene sequence. Each gapmer listed in the tables below is targeted to the KLK3 eRNA sequence represented by SEQ ID NO: 2.

TABLE 1

| Target | Target | | | % | SEQ |
|---|---|---|---|---|---|
| Start Site | Stop Site | ISIS No | Sequence | inhibition | ID NO |
| 408 | 423 | 735245 | GAACCTTGGTTAGGCA | 60 | 3 |

TABLE 2

| Target | Target | | | % | SEQ |
|---|---|---|---|---|---|
| Start Site | Stop Site | ISIS No | Sequence | inhibition | ID NO |
| 408 | 423 | 735245 | GAACCTTGGTTAGGCA | 56 | 3 |

TABLE 3

| Target | Target | | | % | SEQ |
|---|---|---|---|---|---|
| Start Site | Stop Site | ISIS No | Sequence | inhibition | ID NO |
| 1028 | 1043 | 735285 | ATGGTGCTGGCCACAC | 76 | 4 |

TABLE 4

| Target | Target | | | % | SEQ |
|---|---|---|---|---|---|
| Start Site | Stop Site | ISIS No | Sequence | inhibition | ID NO |
| 1028 | 1043 | 735285 | ATGGTGCTGGCCACAC | 71 | 4 |

What is claimed:

1. A compound comprising of a modified oligonucleotide 16 linked nucleobases in length having a nucleobase sequence comprising the sequence recited in SEQ ID NO: 3 (GAACCTTGGTTAGGCA), wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

2. A compound comprising a modified oligonucleotide 16 linked nucleobases in length having a nucleobase sequence comprising the sequence recited in SEQ ID NO: 4 (ATGGTGCTGGCCACAC), wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment; wherein each nucleoside of each wing segment comprises a contrained ethyl (cEt) nucleoside; wherein each internucleoside linkage is a phosphorothioate linkage; and wherein each cytosine is a 5-methylcytosine.

3. A composition comprising the compound of claim 1 or 2 and a pharmaceutically acceptable carrier.

4. A composition comprising a compound of claim 1 or 2, for use in therapy.

5. A method of treating, preventing, or ameliorating cancer in an individual comprising administering to the individual the compound of claim 1 or 2 or composition of claim 3, thereby treating, preventing, or ameliorating the cancer.

6. The method of claim 5, wherein the cancer is prostate cancer.

7. A method of inhibiting expression of KLK3 eRNA in a cell comprising contacting the cell with the compound of claim 1 or 2, thereby inhibiting expression of KLK3 eRNA in the cell.

8. The method of claim 7, wherein the cell is in the prostate of an individual.

9. The method of claim 8, wherein the individual has, or is at risk of having, prostate cancer.

10. Use of the compound of claim 1 or 2 for treating, preventing, or ameliorating cancer.

11. The use of claim 10, wherein the cancer is prostate cancer.

12. Use of the compound of claim 1 or 2 in the preparation of a medicament for treating, preventing, or ameliorating cancer.

13. The use of claim 12, wherein the cancer is prostate cancer.


SEQUENCE LISTING (7 sequences)

SEQ ID NO: 1 - DNA, 12001 bp, Homo sapien
ctcaggctctgaggggaaacgcctgaggtctttgagcaaggtcagtcctctgttgcacagtctccctcacagggtcattgtgacgatcaaatgtggtcacgtgtatgaggcaccagcacatgcctggctctggggagtgccgtgtaagtgtatgcttgcactgctgaatggctgggatgtgtcagggattatcttcagcacttacagatgctcatctcatcctcacagcatcactatgggatgggtattactggcctcatttgatggagaaactggctgtggctcagaaaggggggaccactagaccagggacactctggatgctggggactccagagaccatgaccactcaccaactgcagagaaattaattgtggcctgatgtccctgtcctggagagggtggaggtggaccttcactaacctcctaccttgaccctctcttttagggctctttctgacctccaccatgatactaggaccccattgtattctgtaccctcttgactctatgacccccactgcccactgcatccagctgggtcccctcctatctctattcccagctggccagtgcagtctcagtgcccacctgtttgtcagtaactctgaaggggctgacattttactgacttgcaaacaaataagctaactttccagagttttgtgaatgctggcagagtccatgagactcctgagtcagaggcaaaggcttttactgctcacagcttagcagacagcatgaggttcatgttcacattagtacaccttgccccccccaaatcttgtagggtgaccagagcagtctaggtggatgctgtgcacacggggtttgtgccactggtgagaaacctgagattaggaatcctcaatcttatactgggacaacttgcaaacctgctcagcctttgtctctgatgaagatattatcttcatgatcttggattgaaaacagacctactctggaggaacatattgtatcgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggagaattgcctcccaacactgttcagccagagccttccacccttgtctgcaggacagtctcaacgttccaccattaaatacttcttctgtcacatcctgcttatttatgcctaaccaaggttctaggtcccgatcgactgtgtctggcagcactccactgccaaacccagaataaggcagcgctcaggatcccgaaggggcatggctggggatcagaacttctgggtttgagtgaggagtgggtccaccctcttgaatttcaaaggaggaagaggctggatgtgaaggaactgggggagggaaagtgtcagttccgaactcttaggtcaatgagggaggagactggtaaggtcccagctcccgaggtactgatgtgggaatggcctaagaatctcatatcctcaggaagaaggtgctggaatcctgaggggtagagttctgggtatatttgtggcttaaggctctttggcccctgaagggcagaggctggaaccattaggtccagggtttggggtgatagtaatgggatctcttgattcctcaagagtctgaggatcgagggttgcccattcttccatcttgccacctaatccttactccacttgagggtatcaccagcccttctagctccatgaaggtgcccctgggcaagcacaatctgagcatgaaagatgccccagaggccttgggtgtcatccactcatcatccagcatccacactctgagggtgtggccagcaccatgacgtcatgttgctgtgactatccctgcagcgtgcctctccagccacctgccaaccgtagagctgccgacatcctcctctggtgggagtggcctgcatggtgccaggctgaggcctagtgtcagacagggagcctggaatcatagggatccaggactcaaaagtgctagagaatggccatatgtcaccatccatgaaatctcaagggcttctgggtggagggcacagggacctgaacttatgggttttccccaagtctattgctctcccaagtgagtctcccagatacgaggcactgtgccagcatcagccttatctccaccacatcttgtaaaagggactacccagggccctgatgaacaccatggtgtgtacaggagtagggggtggaggcacggactcctgtgaggtcacagccaagggagcatcatcatgggtggggaggaggcaatggacaggcttgagaacggggatgtggttgtatttggttttctttggttagataaagtgctgggtataggattgagagtggagtatgaagaccagttaggatggaggatcagattggagttgggttagagatggggtaaaattgtgcttcggatgagtttgggattgacactgtggaggtggtttgggatggcatggctttgggatggaaatagatttgttttgatgttggctcagacatccttggggattgaactggggatgaagctgggtttgattttggaggtagaagacgtggaagtagctgtcagatttgacagtggccatgagttttgtttgatggggaatcaaacaatgggggaagacataagggttggcttgttaggttaagttgcgttgggttgatggggtcggggctgtgtataatgcagttggattggtttgtattaaattgggttgggtcaggttttggttgaggatgagttgaggatatgcttggggacaccggatccatgaggttctcactggagtggagacaaacttcctttccaggatgaatccggggaagccttaattcacgtgtaggggaggtcaggccactggctaagtatatccttccactccagctctaagatggtcttaaattgtgattatctatatccacctctgtctccctcactgtgcttggagtttacctgatcactcaactagaaacaggggaagattttatcaaattctttttttttttttttttttttttgagacagagtctcactctgttgcccaggctggagtgcagtggcgcagtctcggctcactgcaacctctgcctcccaggttcaagtgattctcctgcctcagcctcctgagttgctgggattacaggcatgcagcaccatgcccagctaatttttgtatttttagtagagatggggtttcaccaatgtttgccaggctggcctcgaactcctgacctggtgatccacctgcctcagcctcccaaagtgctgggattacaggcgtcagccaccgcgcccagccacttttgtcaaattcttgagacacagctcgggctggatcaagtgagctactctggttttattgaacagctgaaataaccaactttttggaaattgatgaaatcttacggagttaacagtggaggtaccagggctcttaagagttcccgattctcttctgagactacaaattgtgattttgcatgccaccttaatctttttttttttttttttaaatcgaggtttcagtctcattctatttcccaggctggagttcaatggcgtgatcacagctcactgtagccttgaactcctggccttaagagattctcctgcttcggtctcccaatagctaagactacagtagtccaccaccatatccagataatttttaaattttttggggggccgggcacagtggctcacgcctgtaatcccaacaccatgggaggctgagatgggtggatcacgaggtcaggagtttgagaccagcctgaccaacatggtgaaactctgtctctactaaaaaaaaaaaaaatagaaaaattagccgggcgtggtggcacacggcacctgtaatcccagctactgaggaggctgaggcaggagaatcacttgaacccagaaggcagaggttgcaatgagccgagattgcgccactgcactccagcctgggtgacagagtgagactctgtctcaaaaaaaaaaaattttttttttttttttgtagagatggatcttgctttgtttctctggttggccttgaactcctggcttcaagtgatcctcctaccttggcctcggaaagtgttgggattacaggcgtgagccaccatgactgacctgtcgtttaatcttgaggtacataaacctggctcctaaaggctaaatattttgttggagaaggggcattggattttgcatgaggatgattctgacctgggagggcaggtcagcaggcatctctgttgcacagatagagtgcacaggtctggagaacaaggagtggggggttattggaattccacattgtttgctgcacgttggattttgaaatgctagggaactttgggagactcatatttctgggctagaggatctgtggaccacaagatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacaatctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagctacagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctccatattgcaacagtcctcactcccacaccaggtccccgctccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccacctcctgagcccctcaatcctatcacagtctactgacttttcccattcagctgtgagtgcccaaccctatcccagagaccttgatgcttggcctcccaatcttgccctaggatacccagatgccaaccagacacctccttcttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccgcttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgttcctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgagcacacgcatgggatgggcctggggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggaggctgagggaggagaattgcttgaacctgggaggtggaggttgcagtgagccgagaccgtgccactgcactccagcctgggtgacagagtgagactccgcctcaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggccaggctacctacccacttggaaacccacgccaaagccgcatctacagctgagccactctgaggcctcccctccccagcggtccccactcagctccaaagtctctctcccttttctctcccacactctatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgtccttttaccctcttcctttttcccttggtttctctcagtttctgtatctgcccttcaccctctcacactgctgtttcccaactcgttgtctgtatttttggcctgaactgtgtcttccccaaccctgtgtttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctaggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgaggggaaaggtgagtggggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggcgggagtgagcaaacacctgccgcaggggaggggagggccctgcggcacctgggggagcagagggaacagcatctggccaggcctgggaggaggggcctagagggcgtcaggagcagagaggaggttgcctggctggagtgaaggatcggggcagggtgcgagagggaagaaaggacccctcctgcagggcctcacctgggccacaggaggacactgcttttcctctgaggagtcaggaactgtggatggtgctggacagaagcaggacagggcctggctcaggtgtccagaggctgccgctggcctccctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtggtttattctggtttgttacattgacaggagacacactgaaatcagcaaaggaaacaggcatctaagtggggatgtgaagaaaacagggaaaatctttcagttgttttctcccagtggggtgttgtggacagcacttaaatcacacagaagtgatgtgtgaccttgtgtatgaagtatttccaactaaggaagctcacctgagccttagtgtccagagtttttattgggggtctgtaggataggcatggagtactggaatagctgaccttaacttctcagacctgaggttcccaagagttcaagcagatacagcatggcctagagcctcagatgtacaaaaacaggcattcatcatgaatcgcactgttagcatgaatcatctggcacggcccaaggccccaggtataccaaggcacttgggccgaatgttccaagggattaaatgtcatctcccaggagttattcaagggtgagccctgtacttggaacgttcaggctttgagcagtgcagggctgctgagtcaaccttttactgtacaggggggtgagggaaagggagaagatgaggaaaccgcctagggatctggttctgtcttgtggccaagtggaccatggggctatcccaagaaggaggaattcagaaataggggaaggttgaggaaggacactgaactcaaaggggatacagtgattggtttatttgtcttctcttcacaacattggtgctggaggaattcccaccctgaggttatgaagatgtctgaacacccaacacatagcactggagatatgagctcgacaagagtttctcagccacagagattcacagcctagggcaggaggacactgtacaccaggcagaatgacatgggaattgcgctcacgattggcttgaagaagcaaggactgtgggaggtgggctttgtagtaacaagagggcagggtgaactctgattcccatgggggaatgtgatggtcctgttacaaatttttcaagctggcagggaataaaacccattacggtgaggacctgtggagggcggctgccccaactgataaaggaaatagccaggtgggggcctttcccattgtaggggggacatatctggc

SEQ ID NO: 2 - DNA, 2170 bp, Homo sapien
tgggacaacttgcaaacctgctcagcctttgtctctgatgaagatattatcttcatgatcttggattgaaaacagacctactctggaggaacatattgtatcgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggagaattgcctcccaacactgttcagccagagccttccacccttgtctgcaggacagtctcaacgttccaccattaaatacttcttctgtcacatcctgcttatttatgcctaaccaaggttctaggtcccgatcgactgtgtctggcagcactccactgccaaacccagaataaggcagcgctcaggatcccgaaggggcatggctggggatcagaacttctgggtttgagtgaggagtgggtccaccctcttgaatttcaaaggaggaagaggctggatgtgaaggaactgggggagggaaagtgtcagttccgaactcttaggtcaatgagggaggagactggtaaggtcccagctcccgaggtactgatgtgggaatggcctaagaatctcatatcctcaggaagaaggtgctggaatcctgaggggtagagttctgggtatatttgtggcttaaggctctttggcccctgaagggcagaggctggaaccattaggtccagggtttggggtgatagtaatgggatctcttgattcctcaagagtctgaggatcgagggttgcccattcttccatcttgccacctaatccttactccacttgagggtatcaccagcccttctagctccatgaaggtgcccctgggcaagcacaatctgagcatgaaagatgccccagaggccttgggtgtcatccactcatcatccagcatccacactctgagggtgtggccagcaccatgacgtcatgttgctgtgactatccctgcagcgtgcctctccagccacctgccaaccgtagagctgccgacatcctcctctggtgggagtggcctgcatggtgccaggctgaggcctagtgtcagacagggagcctggaatcatagggatccaggactcaaaagtgctagagaatggccatatgtcaccatccatgaaatctcaagggcttctgggtggagggcacagggacctgaacttatgggttttccccaagtctattgctctcccaagtgagtctcccagatacgaggcactgtgccagcatcagccttatctccaccacatcttgtaaaagggactacccagggccctgatgaacaccatggtgtgtacaggagtagggggtggaggcacggactcctgtgaggtcacagccaagggagcatcatcatgggtggggaggaggcaatggacaggcttgagaacggggatgtggttgtatttggttttctttggttagataaagtgctgggtataggattgagagtggagtatgaagaccagttaggatggaggatcagattggagttgggttagagatggggtaaaattgtgcttcggatgagtttgggattgacactgtggaggtggtttgggatggcatggctttgggatggaaatagatttgttttgatgttggctcagacatccttggggattgaactggggatgaagctgggtttgattttggaggtagaagacgtggaagtagctgtcagatttgacagtggccatgagttttgtttgatggggaatcaaacaatgggggaagacataagggttggcttgttaggttaagttgcgttgggttgatggggtcggggctgtgtataatgcagttggattggtttgtattaaattgggttgggtcaggttttggttgaggatgagttgaggatatgcttggggacaccggatccatgaggttctcactggagtggagacaaacttcctttccaggatgaatccggggaagccttaattcacgtgtaggggaggtcaggccactggctaagtatatccttccactccagctctaagatggtcttaaattgtgattatctatatccacctctgtctccctca

SEQ ID NO: 3 - DNA, 16 bp, Artificial sequence
  Synthetic oligonucleotide
gaaccttggttaggca

SEQ ID NO: 4 - DNA, 16 bp, Artificial sequence
  Synthetic oligonucleotide
atggtgctggccacac

SEQ ID NO: 5 - DNA, 20 bp, Artificial Sequence
  Primer
ggagaattgcctcccaacac

SEQ ID NO: 6 - DNA, 24 bp, Artificial sequence
  Primer
ttaatggtggaacgttgagactgt

SEQ ID NO: 7 - DNA, 24 bp, Artificial Sequence
  Probe
ttcagccagagccttccacccttg